(19) United States
(12) Patent Application Publication
(10) Pub. No.: US 2026/0207932 A1
(43) Pub. Date: Jul. 23, 2026

(54) NERVE STIMULATION SYSTEMS AND METHODS FOR TREATING BLADDER AND/OR BOWEL DYSFUNCTION

(71) Applicant: INSPIRE MEDICAL SYSTEMS, INC., Golden Valley, MN (US)

(72) Inventor: Stephen Lorne Bolea, Excelsior, MN (US)

(73) Assignee: INSPIRE MEDICAL SYSTEMS, INC., Golden Valley, MN (US)

(21) Appl. No.: 19/144,281

(22) PCT Filed: Dec. 29, 2023
(86) PCT No.: PCT/US2023/086495

§ 371 Date: Jun. 27, 2025

Related U.S. Application Data
(60) Provisional application No. 63/436,499, filed on Dec. 31, 2022.

(51) Int. Cl. A61N 1/36; A61N 1/05

(52) U.S. Cl. CPC A61N 1/36007; A61N 1/0514; A61N 1/0556; A61N 1/36062; A61N 1/36135

(57) ABSTRACT

Systems and methods for treating bladder and/or bowel dysfunction of a patient includes implanting a lead carrying one or more stimulation elements to apply stimulation energy to one or more target sites. In some examples, the target site(s) can be one or more of the hypogastric nerve, the pudendal nerve, the pelvic nerve, the external urethral sphincter, and the internal urethral sphincter.

A portion of the population suffers from bladder and/or bowel dysfunction, such as one or both of urinary incontinence (or bladder incontinence) and fecal incontinence (or bowel incontinence). Diet, training, slings, and drug therapies may fail to treat incontinence.

### BRIEF DESCRIPTION OF DRAWINGS

FIG. 1 is a schematic illustration of anatomy of a human pelvic region.

FIG. 2 is a schematic illustration of the pelvic region of FIG. 1 and various nerves.

FIG. 3 is a block diagram of a treatment system in accordance with principles of the present disclosure.

FIG. 4 illustrates a treatment system implanted to a patient in accordance with principles of the present disclosure.

FIG. 5 illustrates another treatment system implanted to a patient in accordance with principles of the present disclosure.

FIG. 6 illustrates another treatment system implanted to a patient in accordance with principles of the present disclosure.

FIG. 7 illustrates another treatment system implanted to a patient in accordance with principles of the present disclosure.

FIG. 8 illustrates another treatment system implanted to a patient in accordance with principles of the present disclosure.

FIG. 9 illustrates another treatment system implanted to a patient in accordance with principles of the present disclosure.

FIG. 10 illustrates another treatment system implanted to a patient in accordance with principles of the present disclosure.

FIG. 11 illustrates another treatment system implanted to a patient in accordance with principles of the present disclosure.

FIG. 12 is a block diagram schematically representing a care engine of a control portion.

FIG. 13 is a diagram schematically representing a patient's body, implantable components, and/or external elements of an example device and/or for use in an example method.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense. It is to be understood that features of the various examples described herein may be combined, in part or whole, with each other, unless specifically noted otherwise.

At least some examples of the present disclosure are directed to implantable devices for diagnosis, therapy, and/or other care of medical conditions. At least some examples may comprise implantable devices and/or methods of implanting devices useful for treating bladder or bowel dysfunctions, including one or both of urinary incontinence and fecal incontinence of a patient, or other pelvic disorders. At least some such examples comprise implanting an electrode to deliver a nerve-stimulation signal to one or more nerves or nerve branches to activate a corresponding external sphincter, such as a branch of the pudendal nerve that activates the external urethral sphincter and/or the external anal sphincter. In some embodiments, operation of the implantable device is controlled in response to sensed information of the patient.

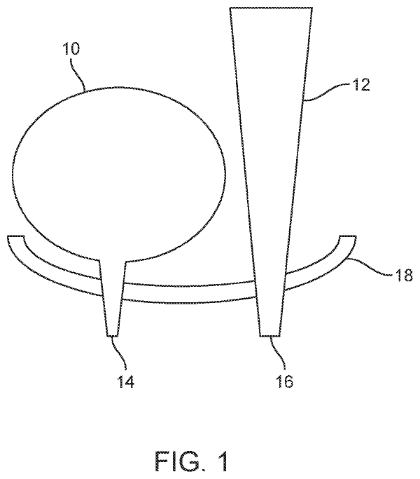

With reference to the greatly simplified view of FIG. 1, the human pelvic region includes a bladder 10 and a rectum 12. Contents of the bladder 10 are evacuated through a urethra 14, whereas contents of the rectum 12 are evacuated through the anus 16. Pelvic floor muscles 18 support the pelvic organs and span the bottom of the pelvis. The pelvic floor muscle layer 18 has holes for passage of the urethra 14 and the anus 16, and normally wraps quite firmly around these holes to help keep the passages shut.

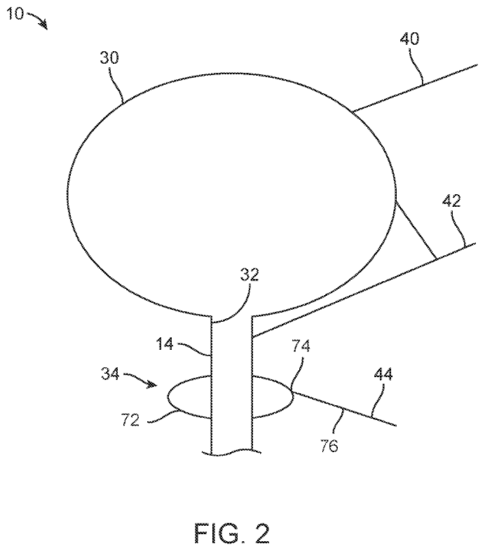

With additional references to the greatly simplified view of FIG. 2, the bladder 10 is a hollow muscular organ connected to the kidneys by the ureters. The detrusor 30 muscle (referenced generally) is smooth muscle found in the wall of the bladder 10. The urethra 14 is a tube or duct by which urine is conveyed out of the body from the bladder 10. Internal and external sphincters control flow of urine through the urethra 14; under normal conditions, when either of these muscles contracts, the urethra 14 is sealed shut. In particular, an internal urethral sphincter (IUS) 32 (referenced generally) is a smooth muscle that constricts the internal orifice of the urethra 14. The IUS 32 is located at the junction of the urethra 14 with the bladder 10 and is continuous with the detrusor muscle 30, but is anatomically and functionally fully independent from the detrusor muscle 30. An external urethral sphincter (EUS) 34 is located in the deep perineal pouch, at the bladder's 10 distal inferior end around the mid urethra in females and inferior to the prostate in males. Urine is excreted from the kidneys and stored in the bladder 10 before elimination via the urethra 14 during what is known as the micturition reflex. During periods of bladder filling, the storage of urine is promoted by the actions of the internal and external urethral sphincters 32, 34 and the pelvic floor musculature 18. During micturition, these sphincters 32, 34 relax and the smooth muscle of the bladder (the detrusor muscle 30) contracts, resulting in the expulsion of urine.

The body of the bladder 10 is directly innervated by efferent fibers that arise from parasympathetic postganglionic neurons in the pelvic ganglia and intramural ganglia and by efferent fibers that arise from sympathetic postganglionic neurons in the lumbosacral sympathetic chain and hypogastric ganglia/pelvic ganglia. This is generally reflected in FIG. 2 by reference to a pelvic nerve 40 and a hypogastric nerve 42. The internal urethral sphincter 32 receives innervation from the hypogastric nerve 42. The external urethral sphincter 34 is directly innervated by motor neurons in the sacral segments of the spinal cord via the pudendal nerve 44.

Urinary continence is generally defined as the act of storing urine in the bladder 10 until the bladder 10 can be appropriately evacuated. Urinary continence requires control of the detrusor muscle 30 and is the result of complex coordination between multiple centers in the brain, brain stem, spinal cord, and peripheral nerves. As described above, micturition is a coordinated act of bladder elimination that involves relaxing the pelvic floor muscles 18, contracting the detrusor muscle 30, and simultaneously opening the urethral sphincters 32, 34 to achieve complete emptying of the bladder. Stress incontinence can be defined as the involuntary leakage of urine from the bladder 10 accompanying physical activity (e.g., laughing, coughing, sneezing, etc.) which places increased pressure on the abdomen. The leakage occurs even though the bladder muscles (detrusor muscle 30) is not contracting and an urge to urinate is not present. Stress incontinence can develop when the urethral sphincters 32, 34, the pelvic floor muscles 18, or all of these structures have been weakened or damaged and cannot dependably hold in urine. With urethral hypermobility, the bladder 10 and urethra 14 shift downward when abdominal pressure rises, and there is no hammock-like support for the urethra 14 to be compressed against to keep it closed. With urethral incompetence, problems in the urinary sphincter 32, 34 keep it from closing fully or allow it to pop open under pressure. Urinary urge incontinence (“UUI”) (sometimes referred to as overactive bladder (“OAB”) or detrusor overactivity) entails the involuntary leakage of urine from the bladder 10 when a sudden strong need to urinate is felt. There is a sudden involuntary contraction of the muscular wall (the detrusor 30) of the bladder that signals an immediate need to urinate, which can happen even when the bladder 10 is not full. Mixed incontinence is the term used to a combination of both overactive bladder and stress incontinence.

Internal and external sphincters are similarly provided with the anus 16 (i.e., the internal anal sphincter and the external anal sphincter), acting to keep the anal canal and orifice closed. Action of the internal anal sphincter (IAS) is entirely involuntary, and it is in a state of continuous maximal contraction. The external anal sphincter (EAS) is always in a state of contraction, but can be voluntarily put into a condition of greater contraction so as to more firmly occlude the anal orifice. Similar to urinary continence, bowel continence is the act of storing feces until an acceptable time and opportunity for elimination. Bowel continence requires competent internal and external sphincters, pelvic floor musculature, and intact neurological pathways. Neurological control of bowel continence is complex and requires coordinated reflex activities from the autonomic and enteric nervous systems. The colon can be visualized as a closed, pliant tube bounded by the ileocecal valve and the anal sphincter. The continuous, smooth muscle layer at the end of the rectum 12 thickens to form the internal anal sphincter (IAS); the external anal sphincter (EAS) is a circular band of striated muscle that contracts with the pelvic floor. Parasympathetic stimulation of the IAS from the pelvic plexus originates from the sacral cord (S1 to S2). Sympathetic stimulation of the IAS causes contraction. The EAS is composed of both smooth and striated muscle. The smooth muscle of the EAS is innervated by the enteric nervous system. The striated component of the EAS is innervated by the pudendal nerve that exits the cord at sacral levels S2, S3, and S4.

Fecal incontinence can be defined as the involuntary loss of rectal contents (feces, gas) through the anal canal and the inability to postpone an evacuation until socially convenient. For example, injuries to one or both of the EAS and IAS may make it difficult to hold stool back properly. Injury to the nerves that sense stool in the rectum or those that control the anal sphincter can also lead to fecal incontinence. A generalized weakness of the pelvic floor 18 can lead to an impaired barrier to stool in the rectum 12 entering the anal canal, and this is associated with incontinence to solids. The pelvic floor 18 is innervated by the pudendal nerve and the S3 and S4 branches of the pelvic plexus. If the pelvic floor muscles 18 lose their innervation, they cease to contract and their muscle fibers are in time replaced by fibrous tissue, which is associated with pelvic floor weakness and incontinence.

With the above in mind, various treatment systems and methods have been disclosed that treat bladder and/or bowel dysfunction (e.g., one or more of urinary incontinence, UUI and fecal incontinence) by supplying stimulation signals to an electrode implanted to apply the stimulation signal to one or more nerves and/or muscles of the patient that, for example, influence the behavior of musculature of the pelvic region of the patient, for example musculature relating to one or both of urinary incontinence and fecal incontinence (e.g., the external urethral sphincter 34, the internal urethral sphincter 32, pelvic floor muscles 18, the external anal sphincter, the internal anal sphincter, etc.). Examples of such systems and methods are provided in PCT Publication No. 2020/243104 (Rondoni, et al.) and PCT Publication No. WO 2022/192726 (Rondoni, et al.) the entire teachings of each of which are incorporated herein by reference.

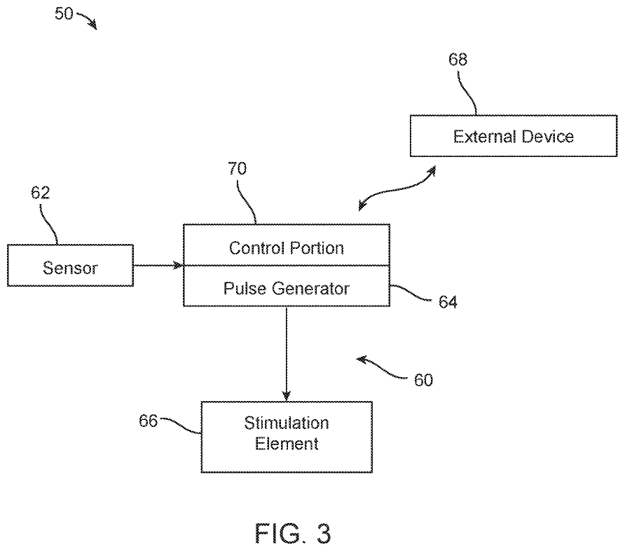

One example of a treatment system 50 for treatment of bladder and/or bowel dysfunction in accordance with principles of the present disclosure is provided in FIG. 3 and includes an implantable medical device (IMD) 60 (referenced generally) and optionally one or more sensors 62 (e.g., one or more of an accelerometer, a pressure sensor, a strain sensor, bioimpedance sensor, etc.). In general terms, the IMD 60 includes an implantable pulse generator or implantable component of a pulse generator (collectively identified as “IPG”) 64 and one or more stimulation elements (e.g., electrode or electrode assembly) 66. The IPG 64 is configured for implantation into a patient, and is configured to provide and/or assist in the performance of therapy to the patient. With formats in which the IPG 64 is an implantable pulse generator, a power source (e.g., battery) is carried within a housing of the implantable pulse generator and from which stimulation energy is generated. With formats in which the IPG 64 is an implantable component of a pulse generator, the implantable component(s) can include a receiver unit (e.g., receiver coil or similar device) that receives a signal from an external device (external the patient) that typically would be positioned on top of the skin over the location of the receiver coil. The external device can generate/deliver the stimulation energy at desired setting (e.g., amplitude, pulse width, frequency, pulse train length, etc.) to be received by the implanted receiver unit and conducted to the stimulation element(s) 66 for activation of tissue. The implanted receiver unit may or may not operate to modify the signal it receives prior to delivery to the stimulation element(s) 66. The external transmitter/controller may receive sensing signals from external sensor, receive sensing signals from the implanted portion of the implantable component via telemetry, etc. Unless stated otherwise, reference to “IPG 64” is inclusive of both an implantable pulse generator and an implantable component of a pulse generator as described above. The stimulation element 66 is configured to be implanted proximate a selected segment or region of the patient's anatomy, and is electrically connected to the IPG 64, for example via a lead. In other embodiments, the IPG 64 and the stimulation element 66 can be provided as components of a single or integral device, such as a microstimulator, as are known in the art. The IPG 64 is programmed to deliver (or is prompted to deliver) stimulation signals to the stimulation element 66 that in turn apply the signal. In some embodiments, the IPG 64 is programmed (or is prompted) to initiate, cease and/or modulate (e.g., titrate) delivered stimulation signals based upon one or more physical parameters of the patient. In this regard, the sensor(s) 62 sense the physical parameter of interest, and signal the so-sensed parameter to the IPG 64 (or other component controlling operation of the IPG 64). The sensor(s) 62 can be carried by the IPG 64, can be connected to the IPG 64, or can be a standalone component not physically connected to the IPG 64. The sensor(s) 62 can be self-contained and can communicate with the IPG 64 in some optional embodiments. In some embodiments, the sensor(s) 62, the IPG 64, and the stimulation element 66 can be provided as components of a single or integral device. In some embodiments, the treatment system 50 can further include an optional external device 68. Where provided, the external device 68 can, in some non-limiting embodiments, wirelessly communicate with the IMD 60.

The IPG 64 can assume various forms known in the art for generating a nerve-stimulating signal for delivery to the stimulation element(s) 66. For example, the IPG 64 can include a sealed case or enclosure maintaining a power source (e.g., battery) and electrical/circuitry components appropriate for formatting energy from the power source as the desired stimulation signal (e.g., a nerve-stimulation signal). In some embodiments, the IPG 64 as provided as part of, or is electronically linked to, a control system that includes a control portion 70 providing one example implementation of a control portion forming a part of, implementing, and/or generally managing stimulation element(s), power/control elements (e.g. pulse generators, microstimulators), sensors, and related elements, devices, user interfaces, instructions, information, engines, elements, functions, actions, and/or methods, as described throughout examples of the present disclosure. In some examples, the control portion 70 includes a controller and a memory. In general terms, the controller comprises at least one processor and associated memories. The controller is electrically couplable to, and in communication with, memory to generate control signals to direct operation of at least some of the stimulation elements, power/control elements (e.g., pulse generators, microstimulators) sensors, and related elements, devices, user interfaces, instructions, information, engines, elements, functions, actions, and/or methods, as described throughout examples of the present disclosure. In some non-limiting examples, these generated control signals include, but are not limited to, employing instructions and/or information stored in the memory to at least direct and manage treatment of bladder and/or bowel dysfunction by stimulating nerve(s), nerve branch(es) and/or muscle(s), for example to activate one or more of the external urethral sphincter 34 and the external anal sphincter, and/or pelvic floor nerves (e.g., the pudendal nerve 44, the sacral nerve) to relax the detrusor muscle 30 and prevent or reduce urgency or frequency.

In some instances, the controller or control portion 70 may sometimes be referred to as being programmed to perform the actions, functions, routines, etc. of the present disclosure. In some examples, at least some of the stored instructions are implemented as, or may be referred to as, a care engine, a sensing engine, monitoring engine, and/or treatment engine. In some examples, at least some of the stored instructions and/or information may form at least part of, and/or, may be referred to as a care engine, sensing engine, monitoring engine, and/or treatment engine.

In response to or based upon commands received via a user interface and/or via machine readable instructions, the controller generates control signals as described above in accordance with at least some of the examples of the present disclosure. In some examples, the controller is embodied in a general purpose computing device while in some examples, the controller is incorporated into or associated with at least some of the stimulation elements, power/control elements (e.g. pulse generators, microstimulators), sensors, and related elements, devices, user interfaces, instructions, information, engines, functions, actions, and/or method, etc. as described throughout examples of the present disclosure.

For purposes of the present disclosure, in reference to the controller, the term “processor” shall mean a presently developed or future developed processor (or processing resources) that executes machine readable instructions contained in a memory. In some examples, execution of the machine readable instructions, such as those provided via the memory of the control portion 70 cause the processor to perform the above-identified actions, such as operating the controller to implement the sensing, monitoring, treatment, etc. as generally described in (or consistent with) at least some examples of the present disclosure. The machine readable instructions may be loaded in a random access memory (RAM) for execution by the processor from their stored location in a read only memory (ROM), a mass storage device, or some other persistent storage (e.g., non-transitory tangible medium or non-volatile tangible medium), as represented by the memory. In some examples, the machine readable instructions may comprise a sequence of instructions, a processor-executable machine learning model, or the like. In some examples, the memory comprises a computer readable tangible medium providing non-volatile storage of the machine readable instructions executable by a process of the controller. In some examples, the computer readable tangible medium may sometimes be referred to as, and/or comprise at least a portion of, a computer program product. In other examples, hard wired circuitry may be used in place of or in combination with machine readable instructions to implement the functions described. For example, the controller may be embodied as part of at least one application-specific integrated circuit (ASIC), at least one field-programmable gate array (FPGA), and/or the like. In at least some examples, the controller is not limited to any specific combination of hardware circuitry and machine readable instructions, nor limited to any particular source for the machine readable instructions executed by the controller.

In some examples, the control portion 70 may be entirely implemented within or by a stand-alone device.

In some examples, the control portion 70 may be partially implemented in the IPG 64 and partially implemented in a computing resource separate from, and independent of, the IPG 64. For instance, in some examples the control portion 70 may be implemented via a server accessible via the cloud and/or other network pathways. In some examples, the control portion 70 may be distributed or apportioned among multiple devices or resources such as among a server, a neurostimulator or neuromodulation treatment device (or portion thereof), and/or a user interface.

In some examples, the control portion 70 is entirely implemented within or by the IPG 64 (thereby defining an IPG assembly), which has at least some of substantially the same features and attributes as a pulse generator (e.g., power/control element, microstimulator) as described throughout the present disclosure. In some examples, the control portion 70 is entirely implemented within or by a remote control (e.g., a programmer) external to the patient's body, such as a patient control and/or a physician control (e.g., the external device 68). In some examples, the control portion 70 is partially implemented in the IPG 64 assembly and partially implemented in the remote control (at least one of the patient control and the physician control).

The systems and methods of the present disclosure are in no way limited to a particular stimulation therapy regimen. The stimulation therapies or algorithms programmed to, or implemented by, the control portion 70 can be of any format deemed useful for the patient being treated, and may or may not act upon information from the sensor(s) 62. With reference between FIGS. 1-3, the system 50 can be configured and implanted to provide stimulation therapy to one or more nerves and/or muscles that, for example, influence the behavior of musculature of the pelvic region of the patient, for example musculature relating to one or both of urinary incontinence and fecal incontinence (e.g., the external urethral sphincter 34, the internal urethral sphincter 32, pelvic floor muscles 18, the external anal sphincter, the internal anal sphincter, etc.). For example, stimulation can be provided to one or more of the pudendal nerve 44, the pelvic nerve 40, the sacral nerve, hypogastric, or branches thereof. For example, stimulation can be provided to a deep branch of the pudendal nerve 44 or other nerve, for example applied to a distal-most branch of the pudendal nerve 44 (or other nerve) at or in highly close proximity to a location where the branch contacts or terminates a muscle (or other anatomical feature) of interest. In other examples, stimulation can be provided to branches from the dorsal genital nerve (DGN) and/or the deep perineal nerve (DPN), the inferior rectal nerve (branch of the pudendal nerve 44), and/or the superficial perineal nerve (also a branch of the pudendal nerve 44). With any of the nerve or other targets of the present disclosure, stimulation therapy can be delivered in a format configured or selected to induce or prevent voiding by the patient. With optional embodiments in which the treatment system 50 is configured and implanted to deliver stimulation to two (or more) target sites (e.g., two or more of the pudendal nerve 44, the pelvic nerve 50, the sacral nerve, the hypogastric nerve, etc., and/or two or more different locations along one incontinence amelioration-related nerve and/or different incontinence amelioration-related nerves, etc.), the so-applied simulation can be toggled (e.g., simultaneous, alternating, overlapping, unilateral, bilateral, selective), optionally while additionally toggling/adjusting one or more stimulation parameters e.g., amplitude, frequency, pulse width, duty cycle, pulse shape, etc.). Alternatively or in addition, the system 50 can apply electrical stimulation to tissue sites proximate a nerve or nerve branch of interest. In yet other embodiments, stimulation can be applied directly to a muscle. Various, non-limiting examples of stimulation protocols or algorithms are described in PCT Publication No. 2020/243104 (Rondoni, et al.) and PCT Publication No. WO 2022/192726 (Rondoni, et al.) the entire teachings of each of which are incorporated herein by reference.

The stimulation element(s) 66 can assume various forms appropriate for applying electrical stimulation to the anatomical feature (e.g., nerve) of interest, and can be provided as part of, or carried by a lead or lead assembly or the like. The stimulation element(s) 66 can be or include one or more electrodes in the form of ring electrodes, segmented electrodes, partial ring electrodes, coil electrodes and helical electrodes. In some examples, the stimulation element(s) may be or include a cuff electrode, comprising at least some of substantially the same features and attributes as described in Bonde et al., U.S. Pat. No. 8,340,785, Self Expanding Electrode Cuff, issued on Dec. 25, 2012 and Bonde et al., U.S. Pat. No. 9,227,053, Self Expanding Electrode Cuff, issued on Jan. 5, 2016, both which are hereby incorporated by reference in their entirety. Moreover, in some examples a stimulation lead, which may comprise one example implementation of a stimulation element, may comprise at least some of substantially the same features and attributes as the stimulation lead described in U.S. Pat. No. 6,572,543 to Christopherson et al., and which is incorporated herein by reference in its entirety. Other non-limiting examples of stimulation elements and leads useful with the present disclosure are provided in PCT Publication No. 2020/243104 (Rondoni, et al.) and PCT Publication No. WO 2022/192726 (Rondoni, et al.) the entire teachings of each of which are incorporated herein by reference.

With the above generalities in mind, the lead can be delivered and implanted in various manners to position the stimulation element(s) 66 at an intended target site. Aspects of the present disclosure provide optional intended target site(s) and optional stimulation therapy formats or techniques. Unless stated otherwise, the stimulation element(s) 66 can be delivered to the intended target site via a variety of different surgical techniques as would be apparent to one of ordinary skill (e.g., locating a device carrying the stimulation element(s), such as a lead, cuff electrode, microstimulator, etc.). In yet other embodiments, the stimulation element(s) can be provided as part of a trialing system that need not necessarily include the sensor(s) 62.

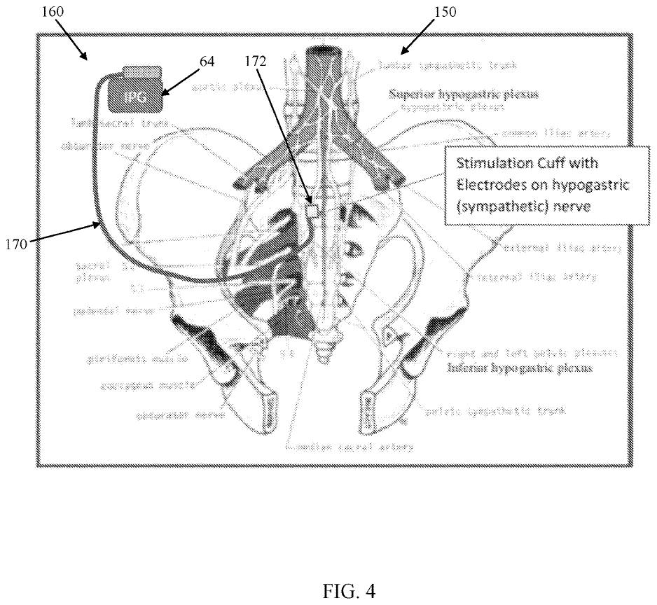

With reference to FIG. 4 (that otherwise illustrates innervation of pelvic organs of a patient 150), some treatments systems and methods of the present disclosure can be formatted to treat urinary incontinence by stimulation of the hypogastric plexus. As a point of reference, the hypogastric plexus is a collection of nerves that is located in front of the fifth lumbar and first sacral vertebral bodies. The hypogastric plexus constitutes the sympathetic pathways that may assist with preventing urine leakage when stimulated. This may be a result of sympathetic activity inhibiting parasympathetic micturition drive. The hypogastric nerve is a continuation of the superior hypogastric plexus; the hypogastric nerve is a sympathetic nerve, carrying inputs from the T12-L3 segments of the spinal cord. The main function of the hypogastric nerve is to connect the superior and inferior hypogastric plexuses, conveying the sympathetic inputs to the inferior hypogastric plexus. The hypogastric nerve passes over the anterior surface of the sacrum and then enters the hypogastric sheath. The fibers then diverge and join the pelvic splanchnic nerves to form the ipsilateral inferior hypogastric plexus. The function of the hypogastric nerve reflects the sympathetic function of the inferior hypogastric plexus. Via the plexus and corresponding branches, the hypogastric nerve provides sympathetic innervation to the rectum and the urinary bladder (as well as other anatomy).

With this mind, a treatment system 160 is shown as implanted within the patient 150. The treatment system 160 can, in many respects, be similar to other treatment systems of the present disclosure, for example the treatment system 50 of FIG. 3 (and described above). In general terms, the treatment system 160 includes the IPG 64 implanted within the patient 150. A lead 170 is connected to the IPG 64 and carries one or more stimulation elements (e.g., electrodes) 172 (referenced generally). The stimulation element(s) 172 can be arranged relative to a body of the lead 170 in various manners, for example as part of a stimulation cuff (as noted in FIG. 4). Other formats that may or may not include a cuff or cuff body are also acceptable. Regardless, the stimulation element(s) 172 are positioned to deliver stimulation energy to, or in close proximity to, the hypogastric plexus, for example at or proximate the superior or inferior hypogastric nerve. The stimulation element(s) 172 can be delivered to, and implanted at, a desired hypogastric plexus target site (e.g., the superior or inferior hypogastric nerve) in various fashions. In some non-limiting examples, the stimulation element(s) 172 can be provided with or carried by a cuff body (that is otherwise included with the lead 170), and the cuff can be delivered to and placed about the target hypogastric plexus nerve via an open or laparoscopic surgical approach. While FIG. 4 reflects a unilateral hypogastric plexus stimulation treatment approach, in other embodiments the system 160 can be configured (e.g., one or more additional leads routed from the IPG 64) to provide bilateral stimulation to the hypogastric plexus (e.g., stimulation elements located to deliver stimulation to the left and right hypogastric nerves). The open or laparoscopic surgical approach can also be utilized with other lead configurations that do not otherwise include a cuff-type body carrying the stimulation element(s) 172.

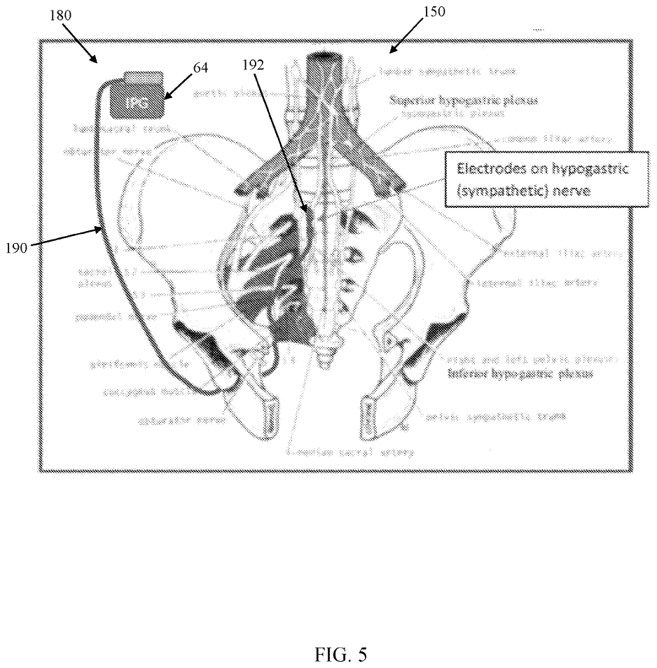

The stimulation element(s) 172 placement and corresponding configurations implicated by FIG. 4 are non-limiting examples of hypogastric plexus stimulation-based bladder and/or bowel dysfunction stimulation treatment systems and methods of the present disclosure. For example, FIG. 5 illustrates another example of a treatment system 180 implanted with the patient 150. The treatment system 180 can, in many respects, be similar to other treatment systems of the present disclosure, for example the treatment system 50 of FIG. 3 (and described above). In general terms, the treatment system 180 includes the IPG 64 implanted within the patient 150. A lead 190 is connected to the IPG 64 and carries one or more stimulation elements (e.g., electrodes) 192 (referenced generally). The stimulation element(s) 192 can be arranged relative to a body of the lead 190 in various manners, for example as electrodes carried by a cylindrical lead body. Other formats are also acceptable. Regardless, the stimulation element(s) 192 are positioned to deliver stimulation energy to, or in close proximity to, the hypogastric plexus, for example at or proximate the superior or inferior hypogastric nerve. The stimulation element(s) 192 can be delivered to, and implanted at, a desired hypogastric plexus target site (e.g., the superior or inferior hypogastric nerve) in various fashions. In some non-limiting examples, the lead 190 can be a percutaneous or stimulation lead that is inserted through the sacral foramen and steered while being advanced to position the stimulation element(s) 192 at a desired location relative to the targeted nerve. In this regard, a desired position of the stimulation element(s) 192 typically entails a location at which stimulation energy delivered or applied by the stimulation element(s) 192 sufficiently “captures” the target nerve (e.g., stimulation capture of the hypogastric plexus). Stimulation capture can be verified in various fashions, for example through sensed electromyography (EMG), sensed electrical nerve activity (ENG), etc. The lead 190 can be fixed to surrounding tissue once a desired placement has been achieved, for example by sutures, self-deploying anchors, tines, etc. While FIG. 5 reflects a unilateral hypogastric plexus stimulation treatment approach, in other embodiments the system 180 can be configured (e.g., one or more additional leads routed from the IPG 64) to provide bilateral stimulation to the hypogastric plexus (e.g., stimulation elements located to deliver stimulation to the left and right hypogastric nerves).

In yet other embodiments, hypogastric plexus stimulation-based bladder and/or bowel dysfunction stimulation treatment systems and methods of the present disclosure can entail stimulation of the superior hypogastric nerve roots or associated spinal cord fibers using a percutaneously placed or surgically placed spinal stimulation lead or dorsal root ganglia stimulating lead.

In yet other embodiments, systems and methods of the present disclosure can entail stimulation of the hypogastric plexus (e.g., stimulation of the hypogastric nerve) in combination with stimulation of one or more additional nerves, such as the pudendal nerve. With these and related embodiments, a combination of pudendal nerve stimulation and hypogastric nerve stimulation can be formatted to provide various forms of treatment, for example to cause contraction of the external urethral sphincter, inhibit relaxation of the internal urethral sphincter, and inhibit detrusor activity.

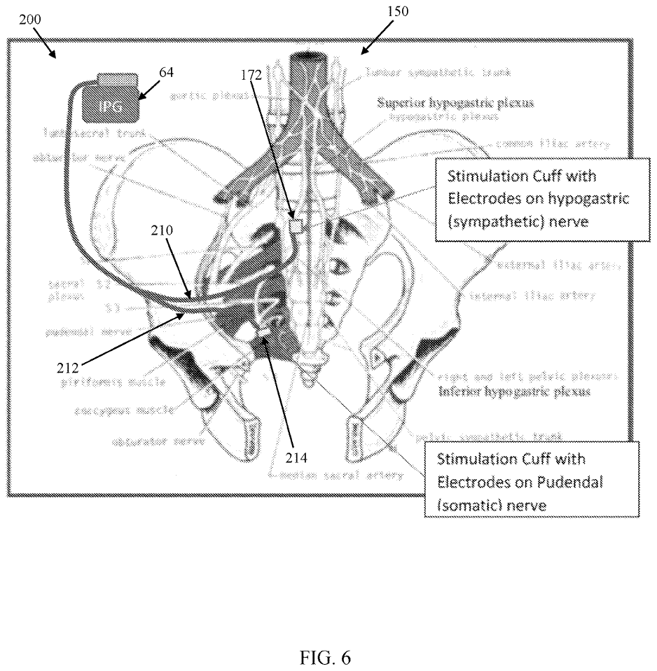

Various treatment system and method configurations can be employed to effect stimulation of the hypogastric plexus and of the pudendal nerve (or other non-hypogastric plexus nerve). For example, FIG. 6 illustrates another treatment system 200 implanted within the patient 150. The system 200 is akin to the system 160 (FIG. 4), and includes the stimulation element(s) 172, carried by a first lead segment 210 in various manners, for example as part of a stimulation cuff. The stimulation element(s) 172 are positioned to deliver stimulation energy to, or in close proximity to, the hypogastric plexus, for example at or proximate the superior or inferior hypogastric nerve. A second lead segment 212 is connected to the IPG 64 and carries one or more second stimulation elements (e.g., electrodes) 214 (referenced generally). The first and second lead segments 210, 212 can be provided as part of a single, bifurcated lead body as shown. Alternatively, the first and second lead segments 210, 212 can be provided as individual lead bodies, each electrically connected to a separate port of the IPG 64. Regardless, the second stimulation element(s) 214 can be arranged relative to a body of the second lead segment 212 in various manners, for example as part of a stimulation cuff (as noted in FIG. 6). Other formats that may or may not include a cuff or cuff body are also acceptable. Regardless, the second stimulation element(s) 214 are positioned to deliver stimulation energy to, or in close proximity to, a desired or target location along the pudendal nerve.

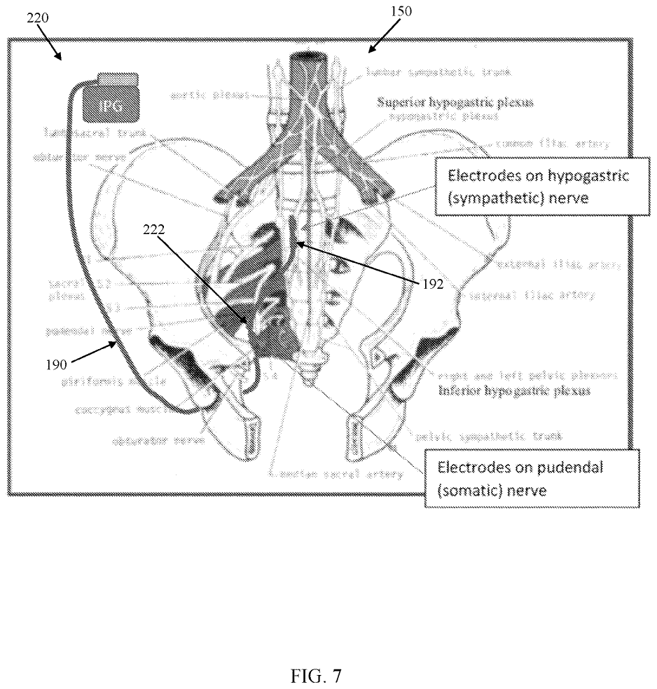

FIG. 7 illustrates another treatment system 220 implanted within the patient 150 and configured for providing pudendal nerve stimulation and hypogastric nerve stimulation. The system 220 is akin to the system 180 (FIG. 4), and includes the stimulation element(s) 192, carried by the lead 190 in various manners, positioned to deliver stimulation energy to, or in close proximity to, the hypogastric plexus, for example at or proximate the superior or inferior hypogastric nerve. The lead 190 further carries one or more second stimulation elements (e.g., electrodes) 222 (referenced generally). In other embodiments, the second stimulation element(s) 222 can be provided with, or carried by, a second lead apart from the lead 190. Regardless, the second stimulation element(s) 222 are positioned to deliver stimulation energy to, or in close proximity to, a desired or target location along the pudendal nerve (e.g., main trunk of the pudendal nerve, deep branch(es) of the pudendal nerve such as the perineal branch(es), directly to one or more muscles of the pelvic floor (e.g., external urethral sphincter, compressor urethrae, levator ani, etc.), etc.

Regardless of how the stimulation elements are delivered to and maintained at the pudendal nerve and the hypogastric nerve (or other hypogastric plexus nerve), various programs or algorithms or models can be employed for delivering stimulation energy. For example, stimulation energy can be delivered to the pudendal nerve and the hypogastric nerve together or simultaneously, coincidently triggered by a sensed parameter, event, or signal (e.g., via the sensor(s) 62 (FIG. 3)) indicative of an event or circumstance of interest. For example, with some programs or algorithms of the present disclosure, the detection of an onset of an event capable of causing stress urinary incontinence event such as increases in intraabdominal pressure can be used or treated as a trigger to deliver stimulation to the hypogastric and pudendal nerves. Alternatively or in addition, the delivery of stimulation energy to the pudendal nerve and the hypogastric nerve (or other hypogastric plexus nerve) can be sequenced such that when one stimulation signal is sent first (such as to stimulate the hypogastric nerve), the second target (such as the pudendal nerve) is delivered either at a time afterwards or after a second level of event detection is achieved, such as a second level of intraabdominal pressure detection (e.g., with some programs or algorithms of the present disclosure, when sensed intraabdominal pressure exceeds a first designated level, stimulation is delivered to the hypogastric nerve; when the sensed intraabdominal pressure later exceeds a second designated level (greater than the first designated level), simulation is further delivered to the pudendal nerve). In other embodiments, the simulation applied to the hypogastric nerve and the pudendal nerve can be toggled (e.g., simultaneous, alternating, overlapping, unilateral, bilateral, selective), optionally while additionally toggling/adjusting one or more stimulation parameters (e.g., amplitude, frequency, pulse width, duty cycle, pulse shape, etc.).

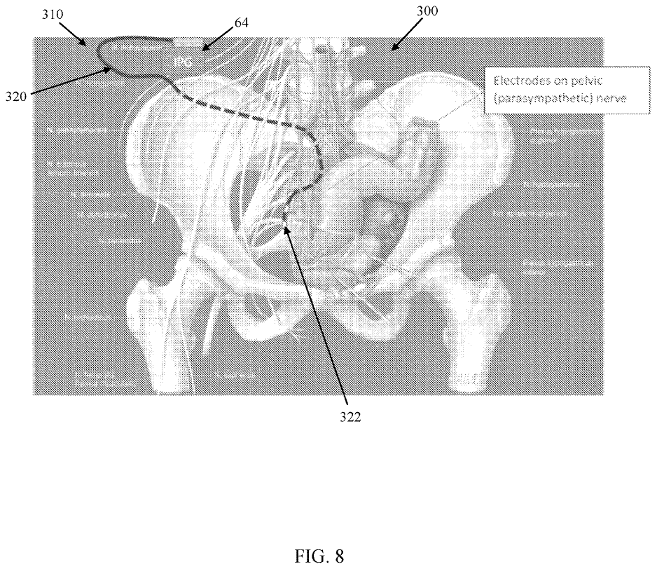

With reference to FIG. 8 (that otherwise illustrates various anatomy of a patient 300), some treatments systems and methods of the present disclosure can be formatted to treat urinary incontinence by stimulation of the pelvic (splanchnic) nerve(s) that otherwise constitute parasympathetic pathways causing voiding. With these and related embodiments, stimulation delivery parameters can be selected to cause nerve blocking sufficient to prevent parasympathetic activity from reaching the detrusor of the bladder or the internal sphincter. As a point of reference, the internal urethral sphincter consists of smooth muscle and is anatomically continuous with the detrusor muscle of the bladder. Because the internal urethral sphincter is composed of smooth muscle, it is not under voluntary control, and is instead controlled through the autonomic nervous system (and more specifically the sympathetic nervous system). During urination, the preganglionic neurons of the sympathetic pathway are inhibited, allowing the muscle to relax.

With this in mind, a treatment system 310 is shown as implanted within the patient 300. The treatment system 310 can, in many respects, be similar to other treatment systems of the present disclosure, for example the treatment system 50 of FIG. 3 (and described above). In general terms, the treatment system 310 includes the IPG 64 implanted within the patient 300. A lead 320 is connected to the IPG 64 and carries one or more stimulation elements (e.g., electrodes) 322 (referenced generally). The stimulation element(s) 322 can be arranged relative to a body of the lead 320 in various manners, for example as electrodes carried by a cylindrical lead body. Other formats are also acceptable. Regardless, the stimulation element(s) 322 are positioned to deliver stimulation energy to, or in close proximity to, the pelvic (splanchnic) nerve along a region constituting a portion of the parasympathetic pathway relating to voiding. The stimulation element(s) 322 can be delivered to, and implanted at, a desired pelvic nerve target site in various fashions. In some non-limiting examples, the lead 320 can be a percutaneous or stimulation lead that is inserted into the third sacral foramen or passing the through the foramen and steered while being advanced to position the stimulation element(s) 322 at a desired location relative to the targeted pelvic nerve. In other embodiments, the stimulation element(s) 322 can be formed as part of, or carried by, a cuff body (e.g., as part of a cuff electrode) that is placed on the pelvic nerve (e.g., via an open or laparoscopic procedure).

Regardless of how the stimulation element(s) are delivered to and maintained at the targeted pelvic nerve, the treatment system 310 can be operated in various fashions to achieve nerve blocking (e.g., via programming to or with the IPG 64). For example, in some embodiments, the IPG 64 can be programmed to deliver high frequency stimulation via the stimulation element(s) 322 sufficient to cause nerve blocking. While FIG. 8 reflects a unilateral stimulation treatment approach, in other embodiments the system 310 can be configured (e.g., one or more additional leads routed from the IPG 64) to provide bilateral stimulation to the left and right pelvic nerves. Alternatively or in addition, the system 310 can be configured to deliver stimulation to discrete or spaced apart regions of a single pelvic nerve.

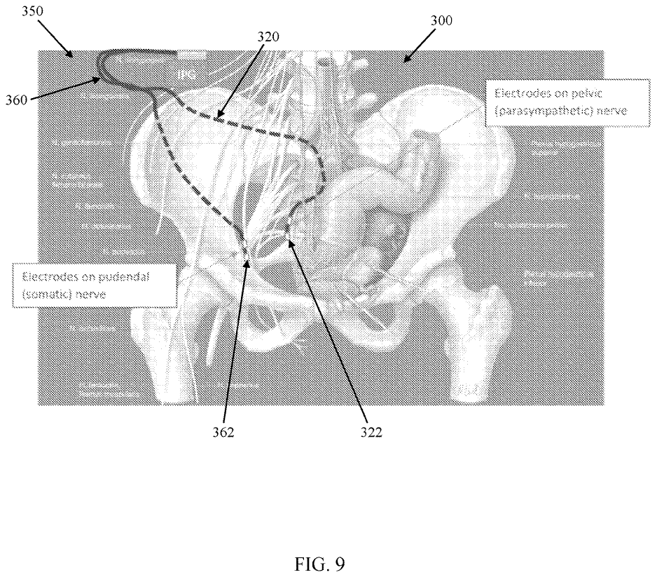

Various treatment system and method configurations can be employed to effect stimulation of the pelvic nerve and one or more additional nerves, such as one or both of the pudendal nerve and the hypogastric nerve (or other non-hypogastric plexus nerve) to prevent urinary leakage. For example, FIG. 9 illustrates another treatment system 350 implanted within the patient 300. The system 350 is akin to the system 310 (FIG. 8), and includes the stimulation element(s) 322, carried by the lead 320 in various manners and positioned to deliver stimulation energy to, or in close proximity to, the pelvic nerve. A second lead 360 is connected to the IPG 64 and carries one or more second stimulation elements (e.g., electrodes) 362 (referenced generally). In other embodiments, a single bifurcated lead can carry the stimulation elements 322, 362. Regardless, with the non-limiting example of FIG. 9, the second stimulation element(s) 362 are to deliver stimulation energy to, or in close proximity to, a desired or target location along the pudendal nerve (e.g., a deep branch of the pudendal nerve). Though not shown, the second stimulation elements 362 can instead be located to apply stimulation energy to a hypogastric nerve. In yet other embodiments, the second stimulation elements 362 are positioned as shown (i.e., targeting the pudendal nerve), and third stimulation element carried by a third lead are located to apply stimulation energy to the hypogastric nerve (or other nerve of the hypogastric plexus).

Regardless of how the stimulation elements are delivered to and maintained at the pelvic nerve, and one or both of the pudendal nerve and the hypogastric nerve (or other hypogastric plexus nerve), various programs or algorithms or models can be employed for delivering stimulation energy to prevent urinary leakage. For example, stimulation energy can be delivered to the pelvic nerve and one or both of the pudendal nerve and the hypogastric nerve together or simultaneously, coincidently triggered by a sensed parameter, event, or signal (e.g., via the sensor(s) 62 (FIG. 3)) indicative of an event or circumstance of interest. For example, with some programs or algorithms of the present disclosure, the detection of an onset of an event capable of causing stress urinary incontinence event such as increases in intraabdominal pressure can be used or treated as a trigger to deliver stimulation to the pelvic nerve and one or both of the pudendal nerve and the hypogastric nerve. Alternatively or in addition, the delivery of stimulation energy to the pelvic nerve and one or both of the pudendal nerve and the hypogastric nerve (or other hypogastric plexus nerve) can be sequenced based on a time of day and/or based on a magnitude of a patient-related parameter. The patient-related parameter can be a sensed parameter (e.g., obtained by one or more sensors installed to the patient) or a non-sensed input. Non-sensed inputs that can provide useful information for determining stimulation timing and/or format can include information on consumption of foods or liquids, activity level, altitude or barometric pressure, GPS, etc. These and other inputs can be collected simply from interfacing with other devices apart from the treatment system such as apps and wearables. Sensor-obtained parameters can assume various forms. For example, with some programs or algorithms of the present disclosure, when sensed intraabdominal pressure exceeds a first designated level, stimulation is delivered to the pudendal nerve alone; when the sensed intraabdominal pressure later exceeds a second designated level (greater than the first designated level), simulation is further delivered to the pelvic nerve and/or the hypogastric nerve. In other embodiments, detected or sensed bladder volume (e.g., using a bioimpedance sensor) can be used to trigger stimulation of the pelvic nerve in a way that provides blocking sufficient to prevent parasympathetic drive while sensed increase in intraabdominal pressure can then be utilized to trigger stimulation of other target sites (e.g., one or both of the pudendal or hypogastric nerve). In other embodiments, the simulation applied to the pelvic nerve and one or both of the pudendal nerve and the hypogastric nerve can be toggled (e.g., simultaneous, alternating, overlapping, unilateral, bilateral, selective), optionally while additionally toggling/adjusting one or more stimulation parameters (e.g., amplitude, frequency, pulse width, duty cycle, pulse shape, etc.).

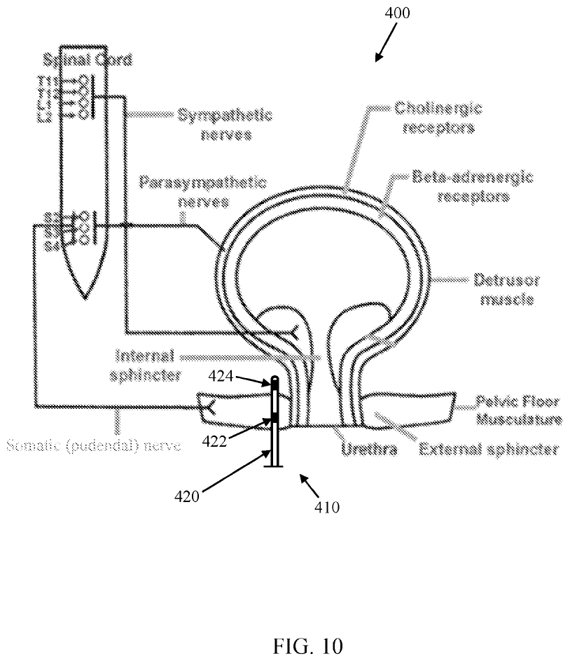

With reference to FIG. 10 (that otherwise diagrammatically illustrates various anatomy of a patient 400), some treatments systems and methods of the present disclosure can be formatted to treat urinary incontinence by stimulation of the external urethral sphincter (or nerve fibers leading to the external urethral sphincter) and the internal urethral sphincter. With this in mind, portions of a treatment system 410 is shown as implanted within the patient 400. The treatment system 410 can, in many respects, be similar to other treatment systems of the present disclosure, for example the treatment system 50 of FIG. 3 (and described above). In general terms, the treatment system 410 includes the IPG (not shown, but akin to the IPG 64 of FIG. 3) implanted within the patient 400. A lead 420 is connected to the IPG and carries one or more first stimulation elements 422 (e.g., electrodes), and one or more second stimulation elements 424 (e.g., electrodes). The stimulation element(s) 422, 424 can be arranged relative to a body of the lead 420 in various manners, for example as electrodes carried by a cylindrical lead body. Other formats are also acceptable.

Upon final implant, the lead 420 has a periurethral placement, arranging the first stimulation element(s) 422 (that are otherwise spaced from a distal tip of the lead 420) proximate the external urethral sphincter (or proximate nerve fibers leading to the external urethral sphincter), and arranging the second stimulation element(s) 424 proximate the internal urethral sphincter. In other words, the lead 420 is arranged to position the first stimulation element(s) 422 such that stimulation energy delivered via the first stimulation element(s) 422 activates the external urethral sphincter; the second stimulation element(s) 424 are positioned such that stimulation energy delivered via the second stimulation element(s) 424 stimulates the internal urethral sphincter, thereby recruiting the sympathetic nerve fibers or smooth muscle tissue of the internal sphincter. The system 410 can be programmed to deliver stimulation energy to one or both of the first and second stimulation element(s) 422, 424 in various manners. In some embodiments, stimulation energy can be simultaneously delivered at the first and second stimulation element(s) 422, 424. In other embodiments, the simulation applied to the external and internal urethral sphincters can be toggled (e.g., simultaneous, alternating, overlapping, unilateral, bilateral, selective), optionally while additionally toggling/adjusting one or more stimulation parameters (e.g., amplitude, frequency, pulse width, duty cycle, pulse shape, etc.).

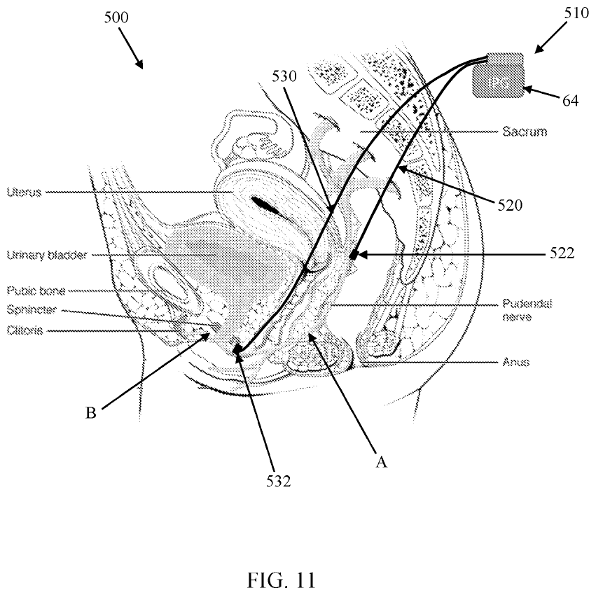

With reference to FIG. 11 (that otherwise illustrates various anatomy of a patient 500), some treatments systems and methods of the present disclosure can be formatted to treat urinary incontinence by selectively applying stimulation at two or more locations along the pudendal nerve. With this in mind, a treatment system 510 is shown as implanted within the patient 500. The treatment system 510 can, in many respects, be similar to other treatment systems of the present disclosure, for example the treatment system 50 of FIG. 3 (and described above). In general terms, the treatment system 510 includes the IPG 64 implanted within the patient 500. A first lead 520 extends from the IPG 64 and carries one or more first stimulation elements 522. The first lead 520 is arranged such that the first stimulation element(s) 522 are located to apply stimulation energy to a trunk of the pudendal nerve. The first stimulation element(s) 522 can be provided with the first lead 520 in various fashions, for example with a cuff body or as part of a cuff electrode. A second lead 530 extends from the IPG 64 and carries one or more second stimulation elements 532. The second lead 530 is arranged such that the second stimulation element(s) 532 are located to apply stimulation energy to a branch of the pudendal nerve, for example a deep perineal branch. The second stimulation element(s) 532 can be provided with the second lead in various fashions, for example as electrode(s) carried by a cylindrical lead body. In other embodiments, the first and second stimulation elements 522, 532 can be carried by or provided with a single lead extending from the IPG 64.

The location of the first stimulation element(s) 522 along to the pudendal nerve implicated by FIG. 11 is but one example. The first stimulation element(s) 522 can be maintained elsewhere along the main trunk of the pudendal nerve; another possible location is indicated by the arrow “A”. Similarly, the location of the second stimulation element(s) 532 along to the pudendal nerve implicated by FIG. 11 is but one example. The second stimulation element(s) 532 can be maintained elsewhere along other branches of the pudendal nerve; another possible location is indicated by the arrow “B”. In yet other embodiments, stimulation elements can be located to apply stimulation energy to two or more branches of the pudendal nerve.

The treatment system 510 can include or operate various programs or algorithms for applying stimulation to the pudendal nerve via the first and second stimulation elements 522, 532. With the arrangement of FIG. 11, the pudendal nerve can be stimulated at multiple locations separately or in combinations together. For example, the pudendal nerve can be stimulated at the main trunk (via the first stimulation element(s) 522) and have greater benefit to the patient through more muscle contraction but have the drawback that unwanted sensory responses result. Simulating the main trunk might then only be effected when, for example, severe levels of intraabdominal pressure are detected; when lower levels of intraabdominal pressure increases are detected only the deep perineal branch is stimulated via the second stimulation element(s) 532, resulting in less unwanted sensory stimulation. In other embodiments, high frequency stimulation or anodal block can be delivered to avoid or reduce unwanted sensations.

As a point of reference, some benefits of functional stimulation (e.g., at levels sufficient to cause muscular contraction) of the pudendal nerve base or posterior pudendal nerve (proximal the pudendal nerve branches such as the perineal branches) can be to achieve a broader reaction to the pelvic floor that could, in turn, help to prevent a leakage event through direct activation of the urethral sphincter, but also by providing support to the bladder, the urethra and involuntary structures. Functional stimulation at this location is also likely to have beneficial effects for bowel incontinence through a similar combination of mechanisms. Sub-functional stimulation as this location has also been shown to have beneficial effects for OAB and bowel incontinence (e.g., non-stress or functional conditions) when stimulated at sub-functional levels. Functional stimulation of the pudendal branches that directly innervate the urethral sphincter muscles (i.e., deep perineal nerve) can also be effective at preventing leak events, however it will have a more localized effect. As compared to stimulation of the pudendal nerve trunk (or “common” pudendal nerve), functional stimulation of the pudendal nerve branches that directly innervate the urethral sphincter muscles may be less effective (though perhaps more comfortable) in some patients because it lacks the broader supportive effects functional stimulation more proximal on the pudendal nerve. Further, stimulation of the pudendal nerve branches that directly innervate the urethral sphincter muscles is not likely to affect bowel incontinence, and is likely to have more limited impact at sub-functional stimulation levels. Stimulation or activation along the posterior pudendal nerve can recruit much of the pelvic floor, providing, for example, urinary stress incontinence and bowel treatment/therapy as well as supporting the pelvic organs to increase the therapeutic effect. In other embodiments, a posterior pudendal nerve target site can serve as an effective location for OAB therapy (e.g., sub-muscle recruitment stimulation).

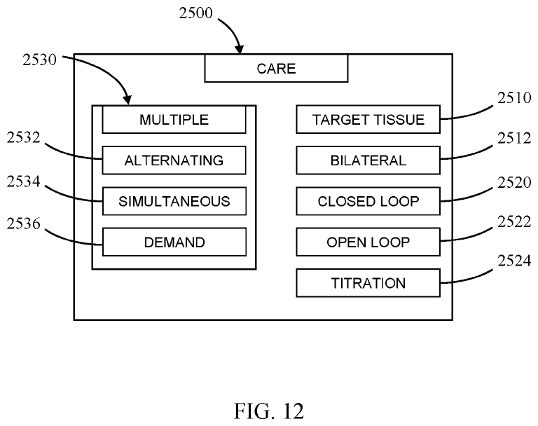

FIG. 12 is a block diagram schematically representing a care engine 2500 of a control portion. In some examples, the care engine 2500 may comprise an example implementation of, and/or at least some of substantially the same features and attributes as, any of the IPGs, care engines and/or the control portions (e.g., FIGS. 3-11) of the present disclosure. Accordingly, the various functions and parameters of the care engine 2500 may be implemented in a manner supportive of, and/or complementary with, the various functions, parameters, portions, etc., of any of the devices and control portions and/or various functions, parameters, portions, etc., relating to stimulation throughout examples of the present disclosure. In some examples, the care engine 2500 may include an implementation of the control portion 70 of FIG. 3.

In some examples, different target tissue may be stimulated using at least one stimulation element. The target tissues may be stimulated at the same time (e.g., simultaneously or overlapping times) or at different times and/or in response to different sensed parameters, such as those described and illustrated in connection with at least FIGS. 3-11.

In some examples, any of the methods, apparatuses, and/or devices may be used to provide bladder and/or bowel dysfunction care to different target tissue, including those described in connection with at least FIGS. 1-11.

As shown by FIG. 12, in some examples, via target tissue parameter 2510, stimulation may be delivered to select target tissue such as, but not limited to, the pudendal nerve, the pelvic nerve, the sacral nerve, hypogastric, or branches thereof. In some examples, target tissues may include any muscles which affect and/or promote continence (e.g., urethral sphincters, detrusor, etc.) and/or nerves which innervate such muscles. In some examples, target tissue includes a combination of nerves and/or muscles such as, but not limited to, terminal fiber ends of nerves where a nerve ending terminates into (or at) the muscle being innervated.

In some examples, in addition to or instead of selecting different tissue for stimulation, the target tissue parameter 2510 may comprise adjusting care parameters (e.g., stimulation parameters) via selecting between (or using a combination of) various locations along a nerve such as stimulating multiple different sites along a particular nerve.

In some examples, in addition to or instead of selecting different nerves for stimulation, the target tissue parameter 2510 may comprise adjusting care parameters via selecting between (or using a combination of) different fascicles within a particular nerve in order to selectively stimulate target efferent fibers while omitting (or minimally impacting) stimulation of other, non-target fibers and/or to selectively stimulate target efferent fibers while omitting (or minimally impacting) stimulation of other, non-target fibers.

In some examples, the care engine 2500 may implement stimulation according to a bilateral parameter 2512 in which stimulation is applied to target tissue on both sides (e.g., left and right) of the patient's body. In some such examples, the bilateral stimulation may be delivered to the same target tissue (e.g., pudendal nerve, pelvic nerve, sacral nerve, hypogastric, or branches thereof) on both sides of the body. However, in some examples, the bilateral stimulation may be delivered to different target tissue or tissue on a left side of the body while stimulating another nerve or tissue on a right side of the body, or vice-versa.

In some examples, the bilateral parameter 2512 may be implemented in a manner complementary with the alternating parameter 2532, simultaneous parameter 2534, or demand parameter 2536 of multiple function 2530, as further described below.

In some examples, the care engine 2500 may comprise a multiple function 2530 by which various care parameters may be implemented in dynamic arrangements. In some such examples, the care engine 2500 may comprise an alternating parameter 2532 by which care provided to one target tissue (e.g., pudendal nerve) may be alternated with care provided to at least one other target tissue (e.g., pelvic nerve). However, the alternating parameter 2532 also may be applied in combination with the bilateral parameter 2512 to apply care to the target tissue (or different target tissue) on opposite sides of the body in which care may be applied on a left side of the body and then applied on the right side of the body in an alternating manner. As used herein, applying or providing care to target tissue may include applying stimulation and/or mechanically maneuvering the target tissue.

In some examples, the care engine 2500 may comprise a simultaneous parameter 2534 by which care may be applied simultaneously to at least two different target tissues. In some examples, the at least two different target tissues comprise two different tissues, such as the pudendal nerve and the pelvic nerve. In some examples, the at least two different target tissues may comprise two different locations along the same tissue or two different fascicles of the same nerve. In some examples, the simultaneous parameter 2534 may apply stimulation per bilateral parameter 2512 simultaneously on opposite sides of the body to the same tissue or different tissue, and/or apply mechanical maneuvering simultaneously on opposite sides of the body to the same tissue.

In some examples, the care engine 2500 may comprise a demand parameter 2536 by which care may be applied to at least one target tissue on a demand basis. For example, stimulation may be applied to one nerve (e.g., pudendal nerve, such as a deep perineal branch thereof) which may be sufficient to achieve the patient metric (e.g., continence) for most circumstances, but may become insufficient for some situations. In the latter situation, to achieve the target patient metric, via the demand parameter 2536, stimulation of a different nerve (e.g., pelvic nerve) may be implemented in addition to, or instead of, stimulation of the first nerve (e.g., pudendal nerve) which was previously being stimulated. In some examples, the first or primary nerve being stimulated may be a nerve other than the pudendal nerve.

In some examples, the care engine 2500 also may further implement at least some aspects of the control portion of FIGS. 3-11 and/or according to at least one of a closed loop parameter 2520, open loop parameter 2522, and titration parameter 2524.

In some examples, the care engine 2500 comprises a closed loop parameter 2520 to deliver care based on sensed patient physiologic information and/or other information (e.g., environmental, temporal, captured by an external system and communicated to the care engine 2500, etc.). In some such examples, via the closed loop parameter 2520 the sensed information may be used to control the particular timing of the care according to bladder fullness information. In some such examples and as previously described, the bladder fullness information and/or other information used with the closed loop parameter 2520 may be determined via the sensors, devices, sensing portions, as previously described in association with at least FIGS. 3-11.

In some examples, the care engine 2500 comprises an open loop parameter (e.g., 2522 in FIG. 12) by which bladder and/or bowel dysfunction care (e.g., “use”) is applied without a feedback loop of sensed physiologic information. In some such examples, in an open loop mode the care is applied during a treatment period without (e.g., independent of) information sensed regarding the patient's bladder fullness, detrusor levels, etc.

In some examples, the care engine 2500 comprises a titration parameter 2524 by which an intensity of the bladder and/or bowel dysfunction therapy may be titrated (e.g., adjusted) to be more intense (e.g., higher stimulation amplitude, greater frequency, and/or greater pulse width) or to be less intense within a treatment period.

In some such examples, the titration parameter 2524 may be implemented according to at least some aspects of the example methods and/or example devices of FIGS. 3-11. Accordingly, in some examples, the titration parameter 2524 may be implemented as automatic titration, while in some examples, the titration parameter may be implemented via manual titration by a patient (or clinician), such as to adjust one or more stimulation parameters. In some examples, the titration parameter may be implemented via combination of patient/manual titration and automatic titration to guide the patient in a manner complementary with manual titration. In some examples, the titration parameter 2524 can entail adjusting a sensitivity of one or more prediction or triggering algorithms according to which stimulation will be delivered in response, for example, to one or more sensed parameters of the patient. For example, the titration parameter 2524 can include a patient selecting a desired sensitivity level (e.g., via the external device 68 (FIG. 3)).

In some examples, at least some aspects of the titration parameter 2524 of the care engine 2500 and/or at least some aspects of titration as generally disclosed throughout FIGS. 3-11 in examples of the present disclosure may comprise (and/or may be implemented) in a manner complementary with and/or via at least some of substantially the same features and attributes as described in: (i) PCT Publication No. 2020/243104 (Rondoni, et al.), and (ii) PCT Publication No. WO 2022/192726 (Rondoni, et al.), each of which are hereby incorporated by reference in their entirety.

The various ranges provided herein include the stated range and any value or sub-range within the stated range. Furthermore, when “about” is utilized to describe a value, this includes, refers to, and/or encompasses variations (up to +/−10%) from the stated value.

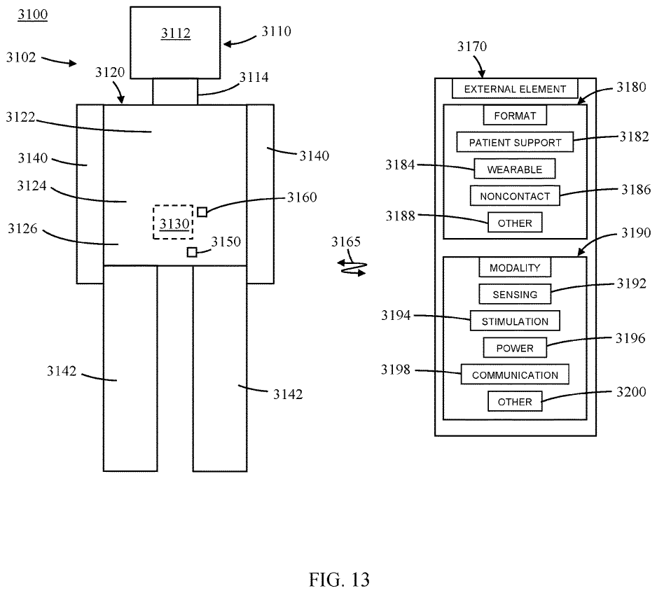

FIG. 13 is a block diagram schematically representing an example arrangement 3100 including patient's body 3102, including example target portions 3110-3142 at which at least some example sensing element(s) and/or stimulation elements may be employed to implement at least some examples of the present disclosure.

As shown in FIG. 13, patient's body 3102 comprises a head-and-neck portion 3110, including head 3112 and neck 3114. As further shown in FIG. 13, the patient's body 3102 comprises a torso 3120, which comprises various organs, muscles, nerves, other tissues, such as but not limited to those in pectoral region 3122 (e.g., lungs, cardiac), abdomen 3124, and/or pelvic region 3126. Organs, muscles, nerves, other tissues of the abdomen 3124 and/or the pelvic region 3126 include bladder 3130, urethra, anus, pelvic floor, etc. As further shown in FIG. 13, the patient's body 3102 comprises limbs such as arms 3140 and legs 3142.

It will be understood that various sensing elements (and/or stimulation elements) as described throughout the various examples of the present disclosure may be deployed within the various regions of the patient's body 3102 to sense and/or otherwise diagnose, monitor, treat various physiologic conditions such as, but not limited to the above-described examples in association with FIGS. 3-12. In some such examples, a stimulation element 3150 may be located in or near the pelvic region 3126 for treating bladder and/or bowel dysfunction (and/or near other nerves/muscles at the same or different location to treat bladder and/or bowel dysfunction and/or other conditions) and/or a sensing element 3160 may be located anywhere within the torso 3120 (or other body regions) to sense physiologic information for providing patient care.

In some examples, at least a portion of the stimulation element 3150 may comprise part of an implantable component/device, such as an IPG whether full sized or sized as a microstimulator. The implantable components (e.g., IPG, other) may comprise a stimulation/control circuit, a power supply (e.g., non-rechargeable, rechargeable), communication elements, and/or other components. In some examples, the stimulation element 3150 also may comprise a stimulation electrode and/or stimulation lead connected to the implantable pulse generator.

Further details regarding a location, structure, operation and/or use of the sensing element 3160, external element(s) 3170, and/or stimulation element 3150 are described above in association with at least FIGS. 3-12.

In some examples, any one of the implantable systems or apparatuses (or a combination thereof) may be implemented as part of the example arrangement 3100 of FIG. 13 instead of, or in addition to (e.g., in complementary relation to), the stimulation element 3150, with at least some examples throughout the disclosure providing further details of such example arrangements. Moreover, at least some aspects (e.g., sensing, control, etc.) associated with an implantable system or apparatus as described in association with FIGS. 3-12 also may be implemented, in whole or part, via external element 3170 of FIG. 13.

In some examples, at least a portion of the stimulation element 3150 may comprise part of an external component/device such as, but not limited to, the external component comprising a pulse generator (e.g., stimulation/control circuitry), power supply (e.g., rechargeable, non-rechargeable), and/other components. In some examples, a portion of the stimulation element 3150 may be implantable and a portion of the stimulation element 3150 may be external to the patient.

Accordingly, as further shown in FIG. 13, the various sensing element(s) 3160 and/or stimulation element(s) 3150 implanted in the patient's body may be in wireless communication (e.g., connection 3165) with at least one external element 3170.

As further shown in FIG. 13, in some examples, the external element(s) 3170 may be implemented via a wide variety of formats such as, but not limited to, at least one of the formats 3180 including a patient support 3182 (e.g., bed, chair, sleep mat, other), wearable elements 3184 (e.g., finger, wrist, head, neck, shirt), noncontact elements 3186 (e.g., watch, camera, mobile device, other), and/or other elements 3188.

As further shown in FIG. 13, in some examples, the external element(s) 3170 may comprise one or more different modalities 3190 such as (but not limited to) a sensing portion 3192, stimulation portion 3194, power portion 3196, communication portion 3198, and/or other portion 3200. The different portions 3192, 3194, 3196, 3198, 3200 may be combined into a single physical structure (e.g., package, arrangement, assembly), may be implemented in multiple different physical structures, and/or with just some of the different portions 3192, 3194, 3196, 3198, 3200 combined together in a single physical structure.

Among other such details, in some examples the external sensing portion 3192 and/or implanted sensing element 3160 may comprise an example implementation of, and/or at least some of substantially the same features and attributes as, the examples further described above in association with FIGS. 3-12.

In some examples, the external stimulation portion 3194 and/or implanted stimulation element 3150 may comprise at least some of substantially the same features and attributes of at least the stimulation arrangements, as further described above in association with at least FIGS. 3-12 and/or other examples throughout the present disclosure.

In some examples, the external power portion 3196 and/or power components associated with implanted stimulation element 3150 may comprise at least some of substantially the same features and attributes of at least the stimulation arrangements, as further described in association with at least FIGS. 3-12 and/or other examples throughout the present disclosure. In some such examples, the respective power portion, components, etc. may comprise a rechargeable power element (e.g., supply, battery, circuitry elements) and/or non-rechargeable power elements (e.g., battery). In some examples, the external power portion 3196 may comprise a power source by which a power component of the implanted stimulation element 3150 may be recharged.

In some examples, the wireless communication portion 3198 (e.g., connection/link at 3165) may be implemented via various forms of radiofrequency communication and/or other forms of wireless communication, such as (but not limited to) magnetic induction telemetry, Bluetooth (BT), Bluetooth Low Energy (BLE), near infrared (NIF), near-field protocols, Wi-Fi, Ultra-Wideband (UWB), and/or other short range or long range wireless communication protocols suitable for use in communicating between implanted components and external components in a medical device environment.

Examples are not so limited as expressed by other portion 3200 via which other aspects of implementing medical care may be embodied in external element(s) 3170 to relate to the various implanted and/or external components described above.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein.

1. A method of treating a bladder and/or bowel dysfunction of a patient, the method comprising:
implanting a first lead into the patient, the first lead carrying at least one first stimulation element; and
applying stimulation energy to a first target site of the patient via the first stimulation element.

2. The method of claim 1, wherein the first target site is a nerve of a hypogastric plexus of the patient.

3. The method of claim 2, wherein the first target site is a sympathetic pathway of the hypogastric plexus.

4. The method of claim 2, wherein the first target site is a hypogastric nerve of the patient.

5. The method of claim 4, wherein the first target site is one of a superior hypogastric nerve and an inferior hypogastric nerve of the patient.

6. The method of claim 4, wherein the step of implanting includes securing the first stimulation element relative to the hypogastric nerve.

7. The method of claim 4, wherein the first stimulation element is carried by a cuff body, and further wherein the step of implanting includes securing the cuff body about the hypogastric nerve.

8. The method of claim 4, wherein the step of implanting includes delivering the first lead into the patient via at least one of an open surgical approach and a laparoscopic surgical approach.

9. The method of claim 4, wherein the first lead includes a cylindrical lead body carrying the first stimulation element, and further wherein the step of implanting includes directing the cylindrical lead body through a sacral foramen of the patient.

10. The method of claim 9, wherein the step of implanting further includes advancing the cylindrical lead body until first stimulation element is located to capture the hypogastric plexus.

11. The method of claim 10, wherein the step of implanting further includes confirming that the first stimulation element has been located to capture the hypogastric plexus by at least one of EMG and ENG sensing.

12. The method of claim 4, wherein the first target site is one of superior hypogastric nerve roots of the patient and spinal cord fibers associated with the superior hypogastric nerve roots.

13. The method of claim 12, wherein the lead is one of a spinal cord stimulation lead and a dorsal root ganglia stimulating lead.

14. The method of claim 4, further comprising:
implanting at least one second stimulation element; and
applying stimulation energy to a second target site of the patient via the second stimulation element.

15. The method of claim 14, wherein the first target site is a left hypogastric nerve of the patient and the second target site is a right hypogastric nerve of the patient.

16. The method of claim 14, wherein the second target site is a pudendal nerve of the patient.

17. The method of claim 16, wherein the steps of applying stimulation energy to the first and second target sites includes causing contraction of an external urethral sphincter of the patient, inhibiting relaxation of an internal urethral sphincter of the patient, and inhibiting activity of a detrusor of the patient.

18. The method of claim 16, wherein the steps of applying stimulation energy to the first and second target sites includes delivering stimulation energy to the first stimulation element and the second stimulation element simultaneously.

19. The method of claim 18, wherein the step of simultaneously delivering of stimulation energy to the first and second stimulation elements is initiated in response to a sensed event.

20. The method of claim 19, wherein the sensed event is an event capable of causing a stress urinary incontinence event.

21. The method of claim 20, wherein the sensed event is an increase in an intraabdominal pressure of the patient.

22. The method of claim 16, wherein the steps of applying stimulation energy to the first and second target sites includes initiating delivery of stimulation energy to the hypogastric nerve via the first stimulation element at a first point in time, and initiating delivery of stimulation energy to the pudendal nerve via the second stimulation element at a second point in time following the first point in time.

23. The method of claim 16, wherein the steps of applying stimulation energy to the first and second target sites includes initiating delivery of stimulation energy to the hypogastric nerve via the first stimulation element in response to a sensed parameter of the patient exceeding a first level, and initiating delivery of stimulation energy to the pudendal nerve via the second stimulation element in response the sensed parameter of the patient exceeding a second level greater than the first level.

24. The method of claim 23, wherein the sensed parameter is intraabdominal pressure.

25. The method of claim 16, wherein the second stimulation element is carried by the first lead.

26. The method of claim 25, wherein the first lead is a bifurcated lead.

27. The method of claim 16, wherein the second stimulation element is carried by a second lead apart from the first lead.

28. The method of claim 1, wherein the first target site is a pelvic (splanchnic) nerve of the patient.

29. The method of claim 28, wherein the pelvic nerve constitutes a parasympathetic pathway causing voiding.

30. The method of claim 29, wherein the step of applying stimulation energy includes formatting the applied stimulation energy to cause nerve blocking of the pelvic nerve.

31. The method of claim 30, wherein the step of applying stimulation energy further includes formatting the applied stimulation energy to prevent parasympathetic activity from reaching at least one of a detrusor and an internal urethral sphincter of the patient.

32. The method of claim 31, wherein the step of applying stimulation energy includes delivering high frequency stimulation.

33. The method of claim 28, wherein the first lead includes a cylindrical lead body carrying the first stimulation element.

34. The method of claim 33, wherein the step of implanting includes inserting the cylindrical lead body into or through a sacral foramen of the patient.

35. The method of claim 34, wherein the sacral foramen is a third sacral foramen of the patient.

36. The method of claim 28, wherein the first stimulation element is carried by a cuff body, and further wherein the step of implanting includes securing the cuff body about the pelvic nerve.

37. The method of claim 36, wherein the step of implanting includes delivering the cuff body into the patient via at least one of an open surgical approach or a laparoscopic surgical approach.

38. The method of claim 28, further comprising:
implanting at least one second stimulation element; and
applying stimulation energy to a second target site of the patient via the second stimulation element.

39. The method of claim 38, wherein the first target site is a left pelvic nerve of the patient and the second target site is a right pelvic nerve of the patient.

40. The method of claim 38, wherein the second target site is one of a pudendal nerve and a hypogastric nerve of the patient.

41. The method of claim 40, further comprising:
implanting at least one third stimulation element; and
applying stimulation energy to a third target site of the patient via the third stimulation element.

42. The method of claim 41, wherein the second target site is a pudendal nerve of the patient and the third target site is a hypogastric nerve of the patient.

43. The method of claim 40, wherein the steps of applying stimulation energy to the first and second target sites includes preventing urinary leakage by the patient.

44. The method of claim 40, wherein the steps of applying stimulation energy to the first and second target sites includes delivering stimulation energy to the first stimulation element and the second stimulation element simultaneously.

45. The method of claim 44, wherein the step of simultaneously delivering of stimulation energy to the first and second stimulation elements is initiated in response to a sensed event.

46. The method of claim 45, wherein the sensed event is an event capable of causing a stress urinary incontinence event.

47. The method of claim 46, wherein the sensed event is an increase in an intraabdominal pressure of the patient.

48. The method of claim 40, wherein the stimulation energy is applied to the first and second target sites in a sequential manner based on at least one of a time delay and a magnitude of a sensed parameter of the patient.

49. The method of claim 40, wherein the steps of applying stimulation energy to the first and second target sites includes initiating delivery of stimulation energy to the pudendal nerve via the second stimulation element in response to a sensed parameter of the patient exceeding a first level, and initiating delivery of stimulation energy to the pelvic nerve via the first stimulation element in response the sensed parameter of the patient exceeding a second level greater than the first level.

50. The method of claim 49, wherein the sensed parameter is intraabdominal pressure.

51. The method of claim 40, wherein the steps of applying stimulation energy to the first and second target sites includes initiating delivery of stimulation energy to the pelvic nerve via the first stimulation element in response to a first sensed parameter of the patient, and initiating delivery of stimulation energy to one of the pudendal nerve and the hypogastric nerve via the second stimulation element in response a second sensed parameter of the patient.

52. The method of claim 51, wherein the first sensed parameter bladder volume and the second sensed parameter is intraabdominal pressure.

53. The method of claim 1, wherein the first target site is one of an external urethral sphincter and nerve fibers leading to the external urethral sphincter of the patient.

54. The method of claim 53, further comprising:
implanting at least one second stimulation element; and
applying stimulation energy to a second target site of the patient via the second stimulation element.

55. The method of claim 54, wherein the second target site is an internal urethral sphincter of the patient.

56. The method of claim 55, wherein the step of applying stimulation energy to the second target site includes recruiting at least one of sympathetic nerve fibers and smooth muscle tissue of the internal urethral sphincter.

57. The method of claim 55, wherein the second stimulation element is carried by the first lead.

58. The method of claim 57, wherein the step of implanting includes securing the first lead in a periurethral location.

59. The method of claim 57, wherein the second stimulation element is located proximate a distal end of the first lead, and the first stimulation element is proximally spaced from the second stimulation element.

60. The method of claim 1, further comprising:
implanting at least one second stimulation element; and
applying stimulation energy to a second target site of the patient via the second stimulation element;
wherein the first and second target sites are along a pudendal nerve of the patient.

61. The method of claim 60, wherein the first target site is a main trunk of the pudendal nerve, and the second target site is a perineal branch of the pudendal nerve.

62. The method of claim 61, wherein the steps of applying stimulation energy to the first and second target sites includes initiating delivery of stimulation energy to the perineal branch of the pudendal nerve via the second stimulation element in response to a sensed parameter of the patient exceeding a first level, and initiating delivery of stimulation energy to the main trunk of the pudendal nerve via the first stimulation element in response the sensed parameter of the patient exceeding a second level greater than the first level.

63. The method of claim 62, wherein the sensed parameter is intraabdominal pressure.

64. The method of claim 60, wherein the second stimulation element is carried by the first lead.

65. The method of claim 60, wherein the second stimulation element is carried by a second lead apart from the first lead.

66. The method of claim 1, wherein the step of applying stimulation energy includes at least one of preventing voiding and inducing voiding by the patient.
